(19) United States
(12) Patent Application Publication
(10) Pub. No.: US 2016/0250429 A1
(43) Pub. Date: Sep. 1, 2016

(54) DEVICE FOR MOUNTING A BREATHING CIRCUIT IN AN ANAESTHESIA MACHINE

(71) Applicant: GENERAL ELECTRIC COMPANY, Schenectady, NY (US)

(72) Inventors: Jiancheng Zhang, WuXi (CN); Hui Fang, WuXi (CN); James Mashak, Madison, WI (US); Ryan Rindy, Madison, WI (US)

(21) Appl. No.: 15/053,630

(22) Filed: Feb. 25, 2016

(30) Foreign Application Priority Data
Feb. 28, 2015 (CN) 201510090367.1

(51) Int. Cl. A61M 16/01; F16M 13/02

(52) U.S. Cl. CPC A61M 16/01; F16M 13/02; A61M 2209/082

(57) ABSTRACT

The present invention relates to a device for mounting a breathing circuit in an anaesthesia machine. The device comprises: a baffle, which is arranged above the breathing circuit; a hanging member, which is arranged on the baffle and the breathing circuit, for hanging the breathing circuit onto the baffle; and a locking member, which is arranged on the breathing circuit or on the baffle, for locking the breathing circuit below the baffle, when the breathing circuit is hung in place.

### CROSS-REFERENCE TO RELATED APPLICATIONS

This application claims priority to Chinese application number 201510090367.1, filed Feb. 28, 2015, the entire disclosures of each of which are hereby incorporated by reference.

### BACKGROUND OF THE INVENTION

The present invention relates to a mounting device, more particularly, to a device for mounting a breathing circuit in an anaesthesia machine.

Generally, a breathing circuit in an anaesthesia machine should be horizontally mounted and connected to a main body of the anaesthesia machine.

An existing device for mounting a breathing circuit to a main body of an anaesthesia machine mainly comprises a plurality of pins and corresponding apertures, wherein the pins are used to bear the weight of the breathing circuit. Because there is unavoidably gaps between the pins and the apertures, causing that after the breathing circuit is mounted in place, the breathing circuit will move under the action of an external force, the user will feel that it is not firm and reliable, and when the user pulls the breathing circuit upwards and downwards, a gas circuit interface and an electric circuit interface connected to the main body of the anaesthesia machine, on the circuit, will loose.

Another existing device for mounting a breathing circuit to a main body of an anaesthesia machine mainly comprises a hook, a latch and a support, wherein these components adopt complex structural designs, to ensure that the breathing circuit is firm and reliable after being mounted to the main body of the anaesthesia machine. However, these complex structural designs add cost.

### BRIEF DESCRIPTION OF THE INVENTION

An embodiment of the present invention provides a device for mounting a breathing circuit in an anaesthesia machine comprising: a baffle, which is arranged above the breathing circuit; a hanging member, which is arranged on the baffle and the breathing circuit, for hanging the breathing circuit onto the baffle; and a locking member, which is arranged on the breathing circuit or on the baffle, for locking the breathing circuit below the baffle, when the breathing circuit is hung in place.

### BRIEF DESCRIPTION OF THE DRAWINGS

The present invention can be better understood in light of the following description of embodiments of the present invention with reference to the accompanying drawings, in which:

FIG. 1 is a schematic view showing a device for mounting a breathing circuit in an anaesthesia machine according to an embodiment of the present invention;

FIG. 2 is a schematic view showing the hanging member in the device according to an embodiment of the present invention;

FIG. 3 is a schematic view showing the locking member in the device according to an embodiment of the present invention;

FIG. 4 is a schematic view showing a process for mounting the breathing circuit onto the main body of the anaesthesia machine by using the device according to the present invention.

### DETAILED DESCRIPTION OF THE INVENTION

Hereafter, a detailed description will be given for preferred embodiments of the present invention. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art relating to the contents disclosed in the present invention, which should not be regarded as insufficient disclosure of the present invention.

Unless defined otherwise, all the technical or scientific terms used in the Claims and the Description should have the same meanings as commonly understood by one of ordinary skilled in the art to which the present invention belongs. The terms “first”, “second” and the like in the Description and the Claims do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

In order to make the purpose, the technical solutions and the advantages of the invention more apparent, the technical solutions of the present invention will be set forth clearly and fully in the following by combining with specific embodiments of the invention and the corresponding accompanying drawings. Obviously, the described embodiments are merely part—not all—of the embodiments in the present invention. In view of the embodiments in the present invention, other embodiments made by one of ordinary skilled in the art without inventive work all fall within the scope of protection of the invention.

The object of the present invention is to provide a device for mounting a breathing circuit in an anaesthesia machine, which achieves a firm connection between the breathing circuit and the main body of the anaesthesia machine, with a simple structure and a lower cost.

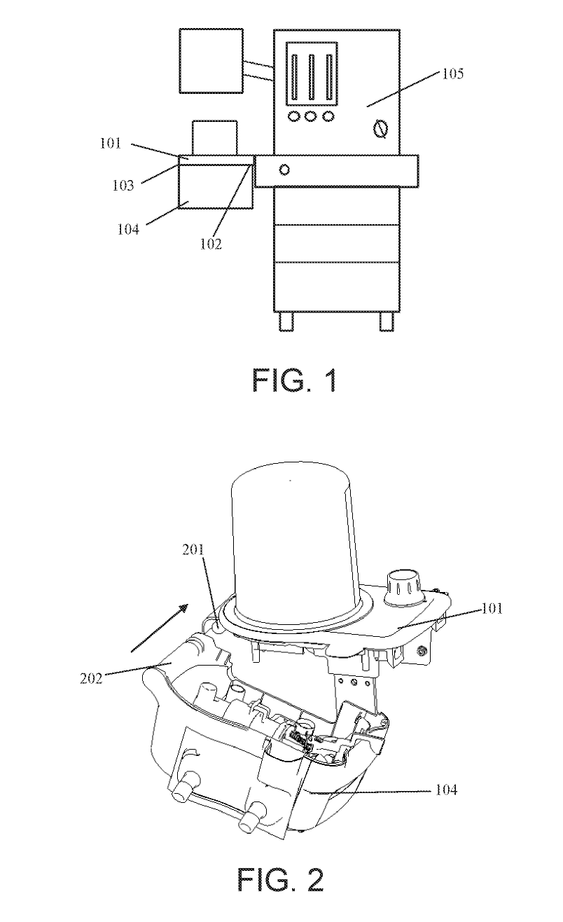

Referring to FIG. 1, FIG. 1 is a schematic view showing a device for mounting a breathing circuit in an anaesthesia machine according to an embodiment of the present invention. The device may comprise a baffle 101, a hanging member 102, and a locking member 103.

The baffle 101 may be arranged above a breathing circuit 104. In an embodiment of the present invention, the baffle 101 may be horizontally fixed to a main body of an anaesthesia machine 105 by a nut. In another embodiment of the present invention, the baffle 101 may be formed by horizontally extending the main body of the anaesthesia machine 105. In other words, the baffle 101 and the main body of the anaesthesia machine 105 are integrally formed. In an embodiment of the present invention, the baffle 101 may be made of die cast, and a plastic plate may be covered onto the die cast.

The hanging member 102 may be arranged on the baffle 101 and the breathing circuit 104, for hanging the breathing circuit 104 onto the baffle 101. Referring to FIG. 2, FIG. 2 is a schematic view showing the hanging member 102 in the device according to an embodiment of the present invention. The hanging member 102 as shown in FIG. 2 may further comprise a supporting member 201 and a hollow member 202. The supporting member 201 may be arranged on the baffle 101, and run parallel to the ground where the anaesthesia machine is located, for providing the breathing circuit 104 with a supporting force. The hollow member 202 may be arranged on the breathing circuit 104, for inserting the supporting member 201 therein (in a direction as shown by the arrow in FIG. 2). In an embodiment of the present invention, the supporting member 201 may be arranged at the rear of the baffle 101, and the hollow member 202 may be arranged at the rear of the breathing circuit 104, correspondingly. The rear referred herein, means that when an operator of the anaesthesia machine is located directly in front of the anaesthesia machine (the right side position in FIG. 2), the poison on the baffle 101 and the breathing circuit 104 which is away from the operator. In an embodiment of the present invention, the cross section of the supporting member 201 may be circular, and the cross section of the hollow member 202 may be semicircular. In an embodiment of the present invention, the supporting member 201 may be a relative longer metal post, and the hollow member 202 may be a hollow area for inserting the supporting member 201 therein. The hollow member 202 may also be a planar area which can be pressed onto the supporting member 201.

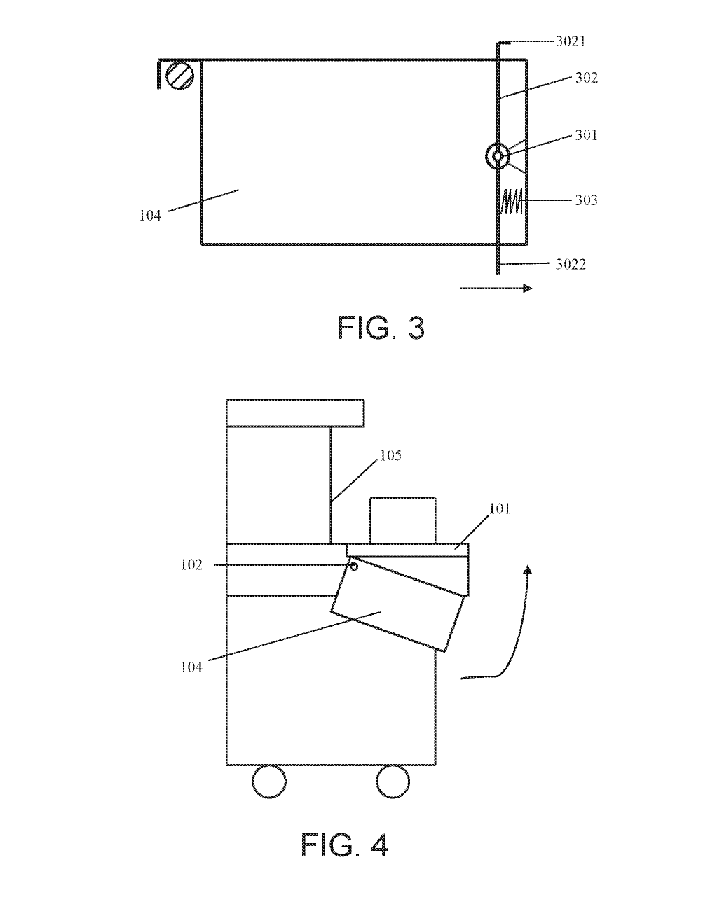

The locking member 103 may be arranged on the breathing circuit 104 or on the baffle 101, for locking the breathing circuit 104 below the baffle 101, when the breathing circuit 104 is hung in place. Referring to FIG. 3, FIG. 3 is a schematic view showing the locking member in the device according to an embodiment of the present invention. In FIG. 3, the locking member 103 is mainly arranged on the breathing circuit 104, and the locking member 103 may further comprise a fulcrum 301, a rotational rod 302 and a spring 303. The fulcrum 301 may be fixed to the breathing circuit 104. The rotational rod 302 may be arranged on the breathing circuit 104 and can rotate about the fulcrum 301, a hook 3021 for locking the breathing circuit 104 onto the baffle 101 may be provided at one end of the rotational rod 302, and a handle 3022 for unlocking the breathing circuit 104 from the baffle 101 may be provided at the other end of the rotational rod 302. One end of the spring 303 may be connected to the breathing circuit 104, and the other end of the spring 303 may be connected to the rotational rod 302.

Referring to FIG. 4, and in conjunction with FIGS. 2, 3, FIG. 4 is a schematic view showing a process for mounting the breathing circuit onto the main body of the anaesthesia machine by using the device according to the present invention. When it is necessary to mount the breathing circuit 104 onto the main body of the anaesthesia machine, the supporting member 201 may be firstly inserted into the hollow member 202 below the baffle, then the breathing circuit 104 is rotated towards the horizontal position (in a direction as shown by the arrow in FIG. 4), and after being rotated to the horizontal position, the locking member 103 may lock the breathing circuit 104 onto the baffle 101, for example: after hooking into a hooking aperture of the baffle 101, the hook 3021 of the locking member 103, under the action of the spring 303, keeps a firmly locked condition, so as to lock the breathing circuit 104 onto the baffle 101. When it is necessary to dismount the breathing circuit 104 from the baffle 101, it only needs to loosen the locking member 103, for example: the handle 3022 at one end of the rotational rod 302 is pulled in a direction as shown by the arrow in FIG. 3, so that the rotational rod 302 rotates about the fulcrum 301, and the hook 3021 at the other end of the rotational rod 302 exits from the hooking aperture of the baffle 101.

Thus, a device for mounting a breathing circuit in an anaesthesia machine according to an embodiment of the present invention has been described. The device disclosed by the present invention, is provided with a baffle 101 which can bear a relative higher pressure above the breathing circuit, thus, can effectively prevent the influence to the breathing circuit due to an accidental impact from above, at the same time, achieve a firm connection between the breathing circuit and the main body of the anaesthesia machine, and improve an user experience of the anaesthesia machine, with a simple structure and a lower cost.

The advantageous technical effects of the present invention are: it can ensure a firm connection between the breathing circuit and the main body, with a simple structure and a low cost, and prevent the influence to the breathing circuit due to an impact from above the breathing circuit.

The above descriptions are merely embodiments of the invention and are not intended to restrict the scope of the invention. All kinds of variations and modifications could be made to the present invention to those skilled in the art. Any modifications, alternatives and improvements made within the spirit and principles of the present invention shall fall within the scope of the appended claims.

We claim:

1. A device for mounting a breathing circuit in an anaesthesia machine, comprising:
a baffle, which is arranged above the breathing circuit;
a hanging member, which is arranged on the baffle and the breathing circuit, for hanging the breathing circuit onto the baffle; and
a locking member, which is arranged on the breathing circuit or on the baffle, for locking the breathing circuit below the baffle, when the breathing circuit is hung in place.

2. The device according to claim 1, wherein the baffle is horizontally fixed to a main body of the anaesthesia machine.

3. The device according to claim 1, wherein the baffle is formed by horizontally extending a main body of the anaesthesia machine.

4. The device according to claim 1, wherein the hanging member further comprises:
a supporting member, which is arranged on the baffle, and runs parallel to the ground where the anaesthesia machine is located, for providing the breathing circuit with a supporting force; and
a hollow member, which is arranged on the breathing circuit, for inserting the supporting member therein.

5. The device according to claim 3, wherein the supporting member is arranged at the rear of the baffle, and the hollow member is arranged at the rear of the breathing circuit.

6. The device according to claim 3, wherein the cross section of the supporting member is circular, and the cross section of the hollow member is semicircular.

7. The device according to claim 1, wherein the locking member further comprises:
a fulcrum, which is fixed to the breathing circuit;
a rotational rod, which is arranged on the breathing circuit and can rotate about the fulcrum, wherein a hook for locking the breathing circuit onto the baffle is provided at one end of the rotational rod, and a handle for unlocking the breathing circuit from the baffle is provided at the other end of the rotational rod; and
a spring, wherein one end of the spring is connected to the breathing circuit, and the other end of the spring is connected to the rotational rod.
